(19) United States
(12) Patent Application Publication
(10) Pub. No.: US 2026/0033760 A1
(43) Pub. Date: Feb. 5, 2026

(54) AUDITORY SYSTEM AND METHOD FOR TESTING HUMAN PROCESSING SPEED

(71) Applicant: Ralph Holmes Boring Benedict, Amherst, NY (US)

(72) Inventor: Ralph Holmes Boring Benedict, Amherst, NY (US)

(21) Appl. No.: 19/280,410

(22) Filed: Jul. 25, 2025

Related U.S. Application Data
(60) Provisional application No. 63/676,965, filed on Jul. 30, 2024.

(51) Int. Cl. A61B 5/16

(52) U.S. Cl. CPC A61B 5/162

(57) ABSTRACT

A computer-implemented method of testing processing speed, including presenting a first set of instructions to a subject, administering a first test, based on the first set of instructions, to the subject, presenting a second set of instructions to the subject, administering a second test, based on the second set of instructions, to the subject, calculating results of at least one of the first test and the second test, and displaying the results on a graphic user interface (GUI).

### CROSS-REFERENCE TO RELATED APPLICATIONS

This application claims the benefit under 35 U.S.C. § 119 (e) of U.S. Provisional Application No. 63/676,965, filed Jul. 30, 2024, which application is incorporated herein by reference in its entirety.

### FIELD

The present disclosure relates generally to testing for humans, and more particularly, to an auditory system and method for testing human mental speed.

### BACKGROUND

Mental chronometry is the scientific study of processing speed or reaction time on cognitive tasks to infer the content, duration, and temporal sequencing of mental operations. Reaction time (RT; also referred to as “response time”) is measured by the elapsed time between stimulus onset and an individual's response on elementary cognitive tasks (ECTs), which are relatively simple perceptual-motor tasks typically administered in a laboratory setting. Mental chronometry is one of the core methodological paradigms of human experimental, cognitive, and differential psychology, but is also commonly analyzed in psychophysiology, cognitive neuroscience, and behavioral neuroscience to help elucidate the biological mechanisms underlying perception, attention, and decision-making in humans and other species.

Current processing speed tests require memory involvement which may create inaccurate results. Furthermore, current processing speed tests require motor movement such as eye function (i.e., near visual acuity and oculomotor function), and thus may be ineffective on certain patients, such as people with multiple sclerosis (PwMS).

### SUMMARY

According to aspects illustrated herein, there is provided an system and method for testing human mental speed.

According to aspects illustrated herein, there is provided a computer-implemented method of testing processing speed, comprising presenting a first set of instructions to a subject, administering a first test, based on the first set of instructions, to the subject, presenting a second set of instructions to the subject, administering a second test, based on the second set of instructions, to the subject, calculating results of at least one of the first test and the second test, and displaying the results on a graphic user interface (GUI).

In an exemplary embodiment, the step of administering the first test comprises receiving an input, initiating a timer, presenting a first stimulus, receiving a response, and determining if the response is correct. In an exemplary embodiment, the step of administering the first test further comprises determining that the response is correct, and adding a point to a score. In an exemplary embodiment, the step of administering the first test further comprises determining that all stimuli have not been presented, and presenting a second stimulus. In an exemplary embodiment, the step of administering the first test further comprises determining that all stimuli have been presented, and determining if a threshold score has been achieved. In an exemplary embodiment, the step of administering the first test further comprises determining that the threshold score has not been achieved, stopping the timer, and prompting the subject to restart the first test.

In an exemplary embodiment, the stimulus is presented auditorily via a speaker. In an exemplary embodiment, the response is presented auditorily via a microphone. In an exemplary embodiment, the step of administering the second test comprises receiving an input to initiate the second test, initiating a timer simultaneously with the received input, presenting a first auditory stimulus, simultaneously with the received input, to the subject, receiving an auditory response from the subject, determining, based on the second set of instructions and an answer key, if the response is correct, and determining if all stimuli for the second test have been presented. In an exemplary embodiment, the step of administering the second test further comprises determining that the response is correct, and adding an amount to a total score.

In an exemplary embodiment, the step of administering the second test further comprises determining that all stimuli have not been presented, and presenting a second stimulus. In an exemplary embodiment, the step of administering the second test further comprises determining that all stimuli have been presented, and stopping the timer.

In an exemplary embodiment, the step of administering the second test comprises presenting a first stimulus to the subject auditorily via a speaker, receiving a first response to the first stimulus auditorily via a microphone, after receiving the first response, presenting a subsequent stimulus to the subject auditorily via a speaker. In an exemplary embodiment, the step of administering the second test comprises adjusting a rate at which stimuli are presented to the subject based on a speed at which the subject provides a response to each stimuli. In an exemplary embodiment, the first set of instructions and/or the second set of instructions comprise a prompt to provide a response of either YES or NO to a stimulus in the form of a single number.

According to aspects illustrated herein, there is provided a system for testing processing speed, comprising at least one processor, a speaker, a microphone, and at least one computer-readable storage medium storing instructions thereon that, when executed by the at least one processor, cause the system to present a first set of instructions to a subject regarding a first test, receive an first input to initiate a first test based on the first set of instructions, initiate a timer, present a first stimulus to the subject auditorily via the speaker, receive a first response from the subject auditorily via the microphone, determine, based on the first set of instructions and a first answer key, if the first response is correct, after determining that a threshold score has been achieved, present a second set of instructions to the subject regarding a second test, the second set of instructions being different than the first set of instructions, receive a second input to initiate the second test based on the second set of instructions, initiate the timer simultaneously with the received second input, present a second stimulus to the subject auditorily via the speaker, receive a second response from the subject auditorily via the microphone, determine, based on the second set of instructions and a second answer key, if the second response is correct, calculating results of at least one of the first test and the second test, and displaying the results on a graphic user interface (GUI).

In an exemplary embodiment, the system further comprises instructions that, when executed by the at least one processor, cause the system to determine that the first response and/or the second response is correct, and add a point to a score. In an exemplary embodiment, the system further comprises instructions that, when executed by the at least one processor, cause the system to adjust a rate at which stimuli are presented based on a speed at which responses are received. In an exemplary embodiment, the system further comprises instructions that, when executed by the at least one processor, cause the system to determine that the threshold score has not been achieved, stopping the timer, and prompting the subject to restart the first test.

According to aspects illustrated herein, there is provided a method of testing processing speed in humans, comprising presenting a subject with a set of instructions including a rule, receiving an input from the subject to initiate a test, initiating a timer simultaneously with the received input, presenting an auditory stimulus, simultaneously with the received input, to the subject, receiving an auditory response from the subject, and determining, based on the set of instructions and an answer key, if the response is correct.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1 is a functional block diagram illustrating an environment, in accordance with exemplary embodiments of the present disclosure.

FIG. 2 is a flow chart depicting operational steps for testing processing speed, in accordance with exemplary embodiments of the present disclosure.

FIG. 3 is a flow chart depicting operational steps for testing processing speed, in accordance with exemplary embodiments of the present disclosure.

FIG. 4 is a flow chart depicting operational steps for testing processing speed, in accordance with exemplary embodiments of the present disclosure.

FIG. 5 is a block diagram of internal and external components of a computer system, in accordance with an embodiment of the present disclosure.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or a device comprising a second element and a third element.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or a device comprising a second element and a third element. A similar interpretation is intended when the phrase “used in at least one of:” is used herein.

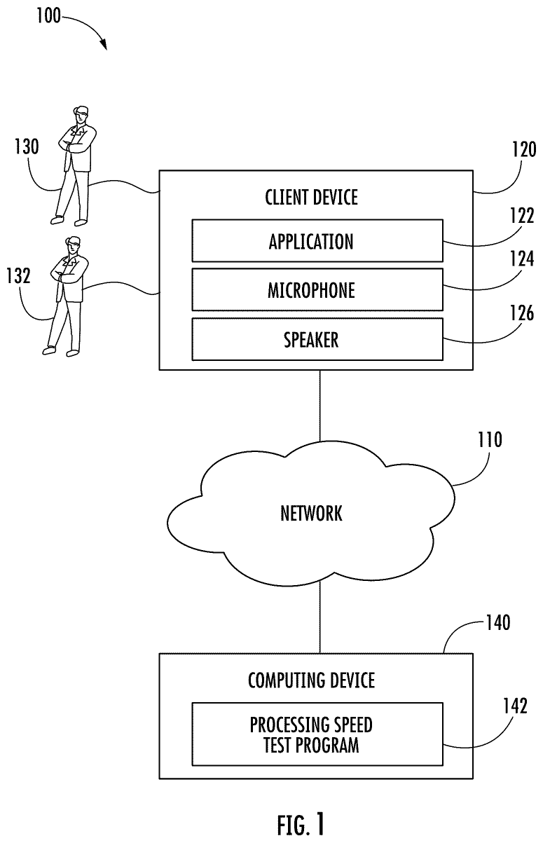

Referring now to the figures, FIG. 1 is a functional block diagram illustrating a processing speed test environment, in accordance with exemplary embodiments of the present disclosure. FIG. 1 provides only an illustration of one implementation, and does not imply any limitations with regard to the environments in which different embodiments may be implemented. Many modifications to the depicted environment may be made by those skilled in the art without departing from the scope of the disclosure as recited by the claims. In an exemplary embodiment, environment 100 includes client device 120. In an exemplary embodiment, environment further includes computing device 140, which may be connected to client device via network 110. In an exemplary embodiment, client device is operated by subject or user 130 and/or administrator 132.

Network 110 can be, for example, a local area network (LAN), a metropolitan area network (MAN), a wide area network (WAN) such as the Internet, or a combination thereof, and can include wired, wireless, or fiber optic connections.

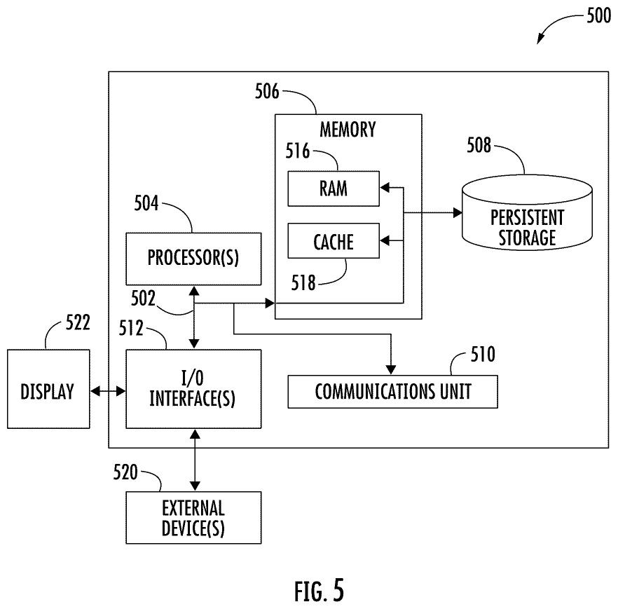

Computing device 140 may be a hardware device that administers one or more processing speed tests, using processing speed test program 142. Computing device 140 is capable of communicating with network 110 and client device 120. In exemplary embodiments, computing device 140 may include a computer. In exemplary embodiments, computing device 140 may include internal and external hardware components, as depicted and described in further detail with respect to FIG. 5.

In exemplary embodiments, processing speed test program 142 is implemented on a web server, which may be a management server, a web server, or any other electronic device or computing system capable of receiving and sending data. The web server can represent a computing system utilizing clustered computers and components to act as a single pool of seamless resources when accessed through a network. The web server may include internal and external hardware components, as depicted and described in further detail with respect to FIG. 5.

Processing speed test program 142 can receive data from multiple entities, for example, client device 120, subject 130, administrator 132, and application 122, and calculate a score for subject 130 based on the results of one or more administered processing speed tests, based on the various methods disclosed herein. Processing speed test program 142 can generally include any software capable of administering one or more processing speed tests and processing results in real time, generating one or more resulting scores, and communicating with client device 120, subject 130, and/or administrator, over network 110.

Client device 120 may be a hardware device capable of communicating with network 110 and computing device 140, and other data sources, for example, via wired connection, wireless connection, or Wi-Fi. In exemplary embodiments, client device 120 may include a computer. In exemplary embodiments, client device 120 may include internal and external hardware components, as depicted and described in further detail with respect to FIG. 5. In an exemplary embodiment, client device 120 comprises an auditory receiving device or microphone 124 operatively arranged to receive input from subject 130 and/or administrator 132. In an exemplary embodiment, client device 120 comprises an auditory transmission device or speaker 136 operatively arranged send transmissions to subject 130 and/or administrator 132.

In exemplary embodiments, client device 120 may include application 122 for facilitating communication (e.g., reading, composing, recording, and/or sending messages (e.g., SMS messages)) between subject 130 and/or administrator 132, and processing speed test program 142. In particular, client device 120 may execute one or more applications (e.g., application 122) for performing the functions of the various embodiments and processes described herein. For example, in some instances, application 122 may be an electronic messaging application (e.g., a messenger, a chat application, a text messenger, etc.). In exemplary embodiments, application 122 may include a messenger application or a messenger within a social media application. In additional embodiments, application 122 may include a skill and/or application of a smart speaker or within a cloud computing service. In further embodiments, application 122 may be a web browser or email provider application. In yet further embodiments, application 122 may be a video chat application. In exemplary embodiments, application 122 may be local to client device 120. In other embodiments, application 122 may be stored and/or at least partially operated via a cloud computing service. In one or more embodiments, application 122 of the client device 120 enables communication with (e.g., sending messages (e.g., text, audio, data, and/or video messages) to and receiving messages from) processing speed test program 142 via the network 110.

In one or more embodiments, application 122 may be a native application installed on client device 120. For example, application 122 may be a mobile application that installs and runs on a mobile device, such as a smart phone or a tablet. In exemplary embodiments, application 122 facilitates the creation of messages, such as SMS messages, to send to other client devices and/or processing speed test program 142. Application 122 may be a text messaging application that is native to client device 120 and/or specific to an operating system of client device 120. Further, application 122 may be independent of processing speed test program 142 (i.e., application 122 may be a generic messaging application not tied to processing speed test program 142 and that does not use processing speed test program 142 to send messages). Alternatively, application 122 may be a client application that is associated with processing speed test program 142 and configured to send messages directly to processing speed test program 142 through the application 122.

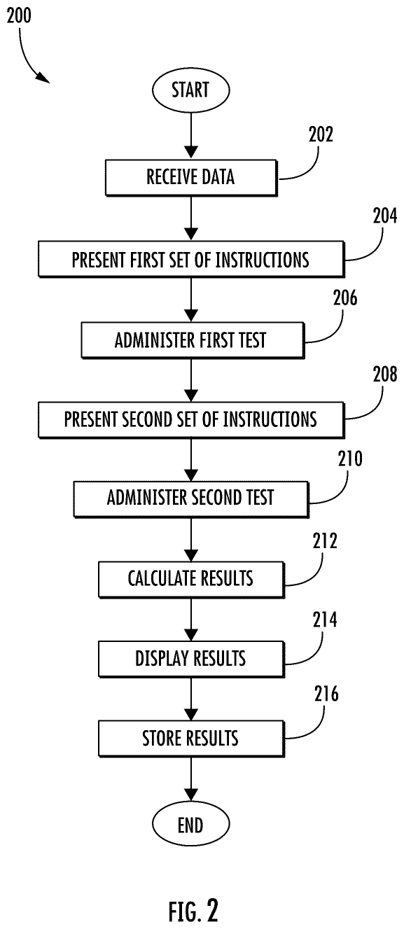

FIG. 2 shows flow chart 200 depicting operational steps for testing processing speed, accordance with exemplary embodiments of the present disclosure. It should be appreciated that with respect to FIG. 2, in exemplary embodiments client device 120 receives data directly or indirectly from subject 130 and/or administrator 132 and sends such data to processing speed test program 142. In an exemplary embodiment, processing speed test program 142 is installed directly on client device 120.

In step 202, processing speed test program 142 receives data or an input from subject 130 and/or administrator 132. Upon beginning the program, subject 130 and/or administrator 132 may be prompted for certain information, to which the subject 130 and/or administer 132 responds. For example, processing speed test program 142 may receive a subject number, an administrator identifier (e.g., initials), subject age, subject sex, subject years of education, test visit number (e.g., first time, second time, etc.), form number (i.e., standard or alternate versions), and/or test version.

In step 204, processing speed test program 142 presents a first set of instructions. In an exemplary embodiment, the first set of instructions are presented in written form on a graphic user interface (GUI) of client device 120. In exemplary embodiments, the first set of instructions are played auditorily via speaker 126. In an exemplary embodiment, the first set of instructions may be read by an examiner. The first set of instructions are related to a first processing speed test. In an exemplary embodiment, the first set of instructions are: YES=1 and NO>1. The first set of instructions may generally recite the following:

You are going to hear a series of numbers played aloud. After each number you hear say “YES” if you hear the number “1” and say “NO” for all other numbers. Do this as quickly as you can without making a mistake. Do you have any questions? We are going to start with a few examples just to make sure you understand the instructions, ready? Press the start button to begin practice, you may correct patient during this practice trail.

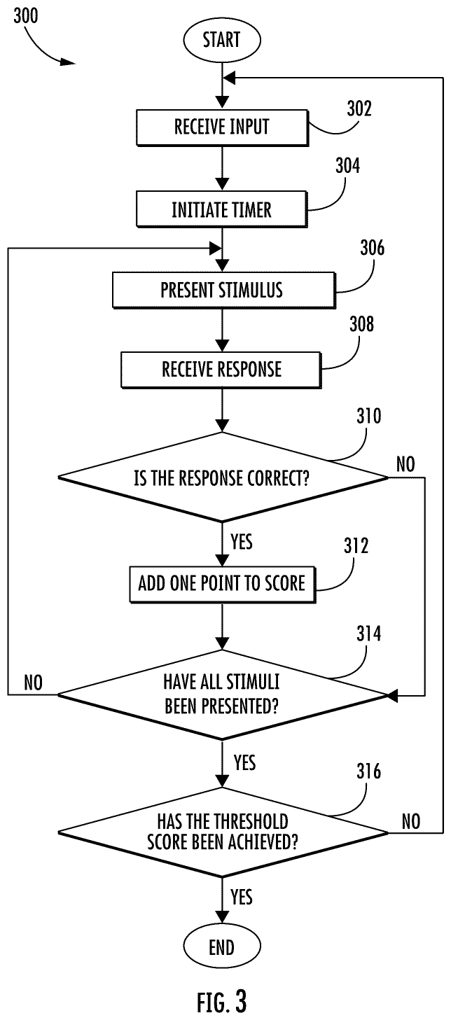

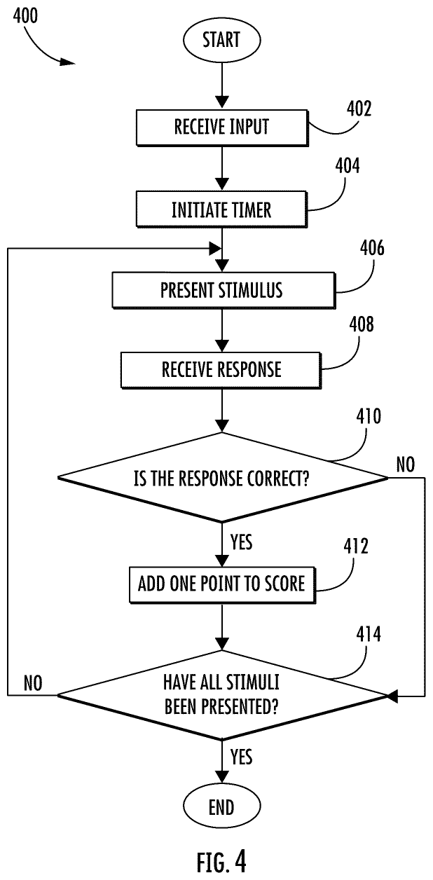

Upon receiving an indication to proceed, processing speed test program 142 then administers a first test. In step 206, processing speed test program 142 administers a first test relating to the first set of instructions. In an exemplary embodiment, the first test is carried out in two parts: first a practice test is administered and then a real test is administered. An exemplary embodiment of the first or practice test is depicted in flow chart 300 of FIG. 3, as will be described in greater detail below. An exemplary embodiment of the second or real test is depicted in flow chart 400 of FIG. 4, as will be described in greater detail below.

In step 208, processing speed test program 142 presents a second set of instructions, different than the first set of instructions. In an exemplary embodiment, the second set of instructions are presented in written form on a GUI of client device 120. In exemplary embodiments, the second set of instructions are played auditorily via speaker 126. The second set of instructions are related to a second processing speed test. In an exemplary embodiment, the second set of instructions are: YES>50 and NO<50. The second set of instructions may generally recite the following:

You are going to hear a series of numbers played aloud. After each number say “YES” if the number you hear is greater than 50 and say “NO” if it's less than 50. Do this as quicky but as accurately as possible. Do you have any questions? We are going to start with a few examples just to make sure you understand the instructions, ready? Press the start button to begin practice, you may correct patient during this practice trial.

Upon receiving an indication to proceed, processing speed test program 142 then administers the second test. In step 210, processing speed test program 142 administers a second test relating to the second set of instructions. In an exemplary embodiment, the second test is carried out in two parts: first a practice test is administered and then a real test is administered.

It should be appreciated that, subsequent to administering the second test in step 210, processing speed test program 142 may issue one or more additional sets of instructions and tests, for example, a third set of instructions, different from the first and second set of instructions, and a third test, and a fourth set of instructions, different from the first, second, and third set of instructions, and a fourth test.

In exemplary embodiments, the third set of instructions are: YES if the number is >50 or any odd number, NO if any other number. The third set of instructions may generally recite the following:

You are going to hear a series of numbers played aloud. After each number you hear say “YES” if the number you hear is greater than 50 OR odd and say “NO” for all other numbers. Do this as quickly but as accurately as possible. Do you have any questions? We are going to start with a few examples just to make sure you understand the instructions, ready? Press the start button to begin practice, you may correct the patient during this practice trial.

In exemplary embodiments, the fourth set of instructions are: YES>50 and ODD, NO<50 or even. The fourth set of instructions may generally recite the following:

You are going to hear a series of numbers played aloud. After each number you hear say “YES” if the number you hear is greater than 50 AND odd and say “NO” for all other numbers. Do this as quickly but as accurately as possible. Do you have any questions? We are going to start with a few examples just to make sure you understand the instructions, ready? Press the start button to begin practice, you may correct the patient during this practice trial.

In step 212, processing speed test program 142 calculates the results of one or more of the tests. For example, processing speed test program 142 may calculate the total number of correct responses, the total time to complete the test (e.g., in seconds), and the percentage of correct responses per test.

In step 214, processing speed test program 142 displays the results to subject 130 and/or administrator 132. In an exemplary embodiment, the results are displayed for each trial completed, for example, displaying total time (e.g., in second) to complete each test, the number of correct responses for each test (e.g., total tally out of a maximum number of stimuli), and the percent of correct responses for each test (e.g., number of correct responses divided by the maximum number of stimuli).

In step 216, processing speed test program 142 stores the results in a database. In an exemplary embodiment, processing speed test program 142 may automatically store the results such that they are correlated with subject 130. In an exemplary embodiment, processing speed test program 142 prompts subject 130 and/or administrator 132 to determine whether to store the results. Based on a response from subject 130 and/or administrator 132 may save or discard the test results. In an exemplary embodiment, all past assessments are saved to a storage device and can be retrieved by subject 130 and/or administrator 132. In an exemplary embodiment, subject 130 and/or administrator 132 can download the results, for example, as a comma-separated values (CSV) file.

FIG. 3 shows flow chart 300 depicting operational steps for testing processing speed, accordance with exemplary embodiments of a practice test.

In step 302, processing speed test program 142 receives an input from subject 130 and/or administrator 132. In an exemplary embodiment the input is an indication to start the test (e.g., button press, auditory command, etc.).

In step 304, processing speed test program 142 initiates a timer.

In step 306, processing speed test program 142 presents a stimulus to subject 130. For example, processing speed test program 142 presents, via speaker 126 or a GUI, a number.

In step 308, processing speed test program 142 receives a response from subject 130. In an exemplary embodiment, the response will be either a YES or NO, and relate to the number presented in the stimulus as well as the corresponding presented set of instructions.

In step 310, processing speed test program 142 determines if the response was correct. In an exemplary embodiment, processing speed test program 142 may utilize voice recognition software to detect the response from the user and compare it to the correct answer. For example, for the set of instructions YES=1 and NO>1, if the stimulus is 1 and the response is YES, then the response is correct. If the stimulus is 1 and the response is NO, then the response is incorrect.

In another example, for the set of instructions YES>50 and NO<50, if the stimulus is 49 and the response is NO, then the response is correct. If the stimulus is 49 and the response is YES, then the response is wrong.

In another example, for the set of instructions YES if the number is >50 or any odd number and NO if any other number, if the stimulus is 17 and the response is NO, then the response is correct. If the stimulus is 18 and the response is YES, then the response is incorrect.

In another example, for the set of instructions YES if the number is >50 and odd, and NO if any other number, if the stimulus is 88 and the response is YES, then the response is incorrect. If the stimulus is 87 and the response is YES, then the response is correct.

If, in step 310, processing speed test program 142 determines that the response is correct, then in step 312 processing speed test program 142 adds one point to a score. Thus, a first correct response will result in a score of one, two correct responses will result in a score of two, etc.

If, in step 310, processing speed test program 142 determines that the response is incorrect, no points are added to the score, and in step 314 processing speed test program 142 determines if all stimuli for the test have been presented. In an exemplary embodiment, for the first test or practice test, a total of ten stimuli are presented.

If, in step 314, processing speed test program 142 determines that all stimuli for the test have not been presented, then the method proceeds to step 306 wherein processing speed test program 142 presents another stimulus.

If, in step 314, processing speed test program 142 determines that all stimuli for the test have been presented, then in step 316 processing speed test program 142 determines if the score meets the threshold score. For example, the threshold score may be 100% in the first or practice test. Additionally, if, in step 314, processing speed test program 142 determines that all stimuli for the test have been presented, then processing speed test program 142 stops the timer.

If, in step 316, processing speed test program 142 determines that the threshold score has not been achieved, then the method restarts and proceed back to step 302.

If, in step 316, processing speed test program 142 determines that the threshold score has been achieved, then the program ends.

FIG. 4 shows flow chart 400 depicting operational steps for testing processing speed, accordance with exemplary embodiments of a practice test.

In step 402, processing speed test program 142 receives an input from subject 130 and/or administrator 132. In an exemplary embodiment the input is an indication to start the test (e.g., button press, auditory command, etc.).

In step 404, processing speed test program 142 initiates a timer.

In step 406, processing speed test program 142 presents a stimulus to subject 130. For example, processing speed test program 142 presents, via speaker 126 or a GUI, a number.

In step 408, processing speed test program 142 receives a response from subject 130. In an exemplary embodiment, the response will be either a YES or NO, and relate to the number presented in the stimulus as well as the corresponding presented set of instructions.

In step 410, processing speed test program 142 determines if the response was correct. In an exemplary embodiment, processing speed test program 142 may utilize voice recognition software to detect the response from the user and compare it to the correct answer.

For example, for the set of instructions YES=1 and NO>1, if the stimulus is 1 and the response is YES, then the response is correct. If the stimulus is 1 and the response is NO, then the response is incorrect. In another example, for the set of instructions YES>50 and NO<50, if the stimulus is 49 and the response is NO, then the response is correct. If the stimulus is 49 and the response is YES, then the response is wrong. In another example, for the set of instructions YES if the number is >50 or any odd number and NO if any other number, if the stimulus is 17 and the response is NO, then the response is correct. If the stimulus is 18 and the response is YES, then the response is incorrect. In another example, for the set of instructions YES if the number is >50 and odd, and NO if any other number, if the stimulus is 88 and the response is YES, then the response is incorrect. If the stimulus is 87 and the response is YES, then the response is correct.

If, in step 410, processing speed test program 142 determines that the response is correct, then in step 412 processing speed test program 142 adds one point to a score. Thus, a first correct response will result in a score of one, two correct responses will result in a score of two, etc.

If, in step 410, processing speed test program 142 determines that the response is incorrect, no points are added to the score, and in step 414 processing speed test program 142 determines if all stimuli for the test have been presented. In an exemplary embodiment, for the second test or real test, a total of twenty stimuli are presented.

If, in step 414, processing speed test program 142 determines that all stimuli for the test have not been presented, then the method proceeds to step 406 wherein processing speed test program 142 presents another stimulus.

If, in step 414, processing speed test program 142 determines that all stimuli for the test have been presented, then the processing speed test program 142 stops the timer and the program ends.

It should be appreciated that processing speed test program 142 is capable of modifying or adjusting the speed of stimuli presentation based on subject 130 response rate. For example, the faster subject 130 responds to a stimulus the faster processing speed test program 142 will present the next stimulus. In this same regard, the slower subject 130 responds to a stimulus the slower processing speed test program 142 will present the next stimulus. Processing speed test program 142 is capable of presenting stimuli following responses with nominal or no delay, thereby achieving a very high level of accuracy when compared to current human issued testing.

FIG. 5 is a block diagram of internal and external components of computing device or computer system 500, which is representative of the computing device of FIG. 1, in accordance with exemplary embodiments of the present disclosure. It should be appreciated that FIG. 5 provides only an illustration of one implementation and does not imply any limitations with regard to the environments in which different embodiments may be implemented. In general, the components illustrated in FIG. 5 are representative of any electronic device capable of executing machine-readable program instructions. Examples of computer systems, environments, and/or configurations that may be represented by the components illustrated in FIG. 5 include, but are not limited to, personal computer systems, server computer systems, thin clients, thick clients, laptop computer systems, tablet computer systems, cellular telephones (i.e., smart phones), multiprocessor systems, microprocessor-based systems, network PCs, minicomputer systems, mainframe computer systems, and distributed cloud computing environments that include any of the above systems or devices.

Computing device 500 includes communications fabric 502, which provides for communications between one or more processing units 504, memory 506, persistent storage 508, communications unit 510, and one or more input/output (I/O) interfaces 512. Communications fabric 502 can be implemented with any architecture designed for passing data and/or control information between processors (such as microprocessors, communications and network processors, etc.), system memory, peripheral devices, and any other hardware components within a system. For example, communications fabric 502 can be implemented with one or more buses.

Memory 506 and persistent storage 508 are computer readable storage media. In this embodiment, memory 506 includes random access memory (RAM) 516 and cache memory 518. In general, memory 506 can include any suitable volatile or non-volatile computer readable storage media. Software is stored in persistent storage 508 for execution and/or access by one or more of the respective processors 504 via one or more memories of memory 506.

Persistent storage 508 may include, for example, a plurality of magnetic hard disk drives. Alternatively, or in addition to magnetic hard disk drives, persistent storage 508 can include one or more solid state hard drives, semiconductor storage devices, read-only memories (ROM), erasable programmable read-only memories (EPROM), flash memories, or any other computer readable storage media that is capable of storing program instructions or digital information.

The media used by persistent storage 508 can also be removable. For example, a removable hard drive can be used for persistent storage 508. Other examples include optical and magnetic disks, thumb drives, and smart cards that are inserted into a drive for transfer onto another computer readable storage medium that is also part of persistent storage 508.

Communications unit 510 provides for communications with other computer systems or devices via a network. In this exemplary embodiment, communications unit 510 includes network adapters or interfaces such as a TCP/IP adapter cards, wireless Wi-Fi interface cards, or 3G, 4G, or 5G wireless interface cards or other wired or wireless communications links. The network can comprise, for example, copper wires, optical fibers, wireless transmission, routers, firewalls, switches, gateway computers and/or edge servers. Software and data used to practice embodiments of the present disclosure can be downloaded to computing device 500 through communications unit 510 (i.e., via the Internet, a local area network, or other wide area network). From communications unit 510, the software and data can be loaded onto persistent storage 508.

One or more I/O interfaces 512 allow for input and output of data with other devices that may be connected to computing device 500. For example, I/O interface 512 can provide a connection to one or more external devices 520 such as a keyboard, computer mouse, touch screen, virtual keyboard, touch pad, pointing device, or other human interface devices. External devices 520 can also include portable computer readable storage media such as, for example, thumb drives, portable optical or magnetic disks, and memory cards. I/O interface 512 also connects to display 522.

Display 522 provides a mechanism to display data to a user and can be, for example, a computer monitor. Display 522 can also be an incorporated display and may function as a touch screen, such as a built-in display of a tablet computer.

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In exemplary embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### REFERENCE NUMERALS

100 Processing speed test environment
110 Network
120 Client device
122 Application
124 Microphone
126 Speaker
130 User or subject
132 Administrator
140 Computing device
142 Processing speed test environment program
200 Flow chart
202 Step
204 Step
206 Step
208 Step
210 Step
212 Step
214 Step
216 Step
300 Flow chart
302 Step
304 Step
306 Step
308 Step
310 Step
312 Step
314 Step
316 Step
400 Flow chart
402 Step
404 Step
406 Step
408 Step
410 Step
412 Step
414 Step
500 Computing device
502 Communications fabric
504 Processing units
506 Memory
508 Persistent storage
510 Communications unit
512 Input/output (I/O) interfaces
516 Random access memory (RAM)
518 Cache memory
520 External device(s)
522 Display

What is claimed is:

1. A computer-implemented method of testing processing speed, comprising:
presenting a first set of instructions to a subject;
administering a first test, based on the first set of instructions, to the subject;
presenting a second set of instructions to the subject;
administering a second test, based on the second set of instructions, to the subject;
calculating results of at least one of the first test and the second test; and
displaying the results on a graphic user interface (GUI).

2. The method as recited in claim 1, wherein the step of administering the first test comprises:
receiving an input;
initiating a timer;
presenting a first stimulus;
receiving a response; and
determining if the response is correct.

3. The method as recited in claim 2, wherein the step of administering the first test further comprises:
determining that the response is correct; and
adding a point to a score.

4. The method as recited in claim 2, wherein the step of administering the first test further comprises:
determining that all stimuli have not been presented; and
presenting a second stimulus.

5. The method as recited in claim 4, wherein the step of administering the first test further comprises:
determining that all stimuli have been presented; and
determining if a threshold score has been achieved.

6. The method as recited in claim 5, wherein the step of administering the first test further comprises:
determining that the threshold score has not been achieved;
stopping the timer; and
prompting the subject to restart the first test.

7. The method as recited in claim 2, wherein the stimulus is presented auditorily via a speaker.

8. The method as recited in claim 2, wherein the response is presented auditorily via a microphone.

9. The method as recited in claim 1, wherein the step of administering the second test comprises:
receiving an input to initiate the second test;
initiating a timer simultaneously with the received input;
presenting a first auditory stimulus, simultaneously with the received input, to the subject;
receiving an auditory response from the subject;
determining, based on the second set of instructions and an answer key, if the response is correct; and
determining if all stimuli for the second test have been presented.

10. The method as recited in claim 9, wherein the step of administering the second test further comprises:
determining that the response is correct; and
adding an amount to a total score.

11. The method as recited in claim 9, wherein the step of administering the second test further comprises:
determining that all stimuli have not been presented; and
presenting a second stimulus.

12. The method as recited in claim 9, wherein the step of administering the second test further comprises:
determining that all stimuli have been presented; and
stopping the timer.

13. The method as recited in claim 1, wherein the step of administering the second test comprises:
presenting a first stimulus to the subject auditorily via a speaker;
receiving a first response to the first stimulus auditorily via a microphone; and
after receiving the first response, presenting a subsequent stimulus to the subject auditorily via a speaker.

14. The method as recited in claim 13, wherein the step of administering the second test comprises:
adjusting a rate at which stimuli are presented to the subject based on a speed at which the subject provides a response to each stimuli.

15. The method as recited in claim 1, wherein the first set of instructions and/or the second set of instructions comprise a prompt to provide a response of either YES or NO to a stimulus in the form of a single number.

16. A system for testing processing speed, comprising:
at least one processor;
a speaker;
a microphone; and
at least one computer-readable storage medium storing instructions thereon that, when executed by the at least one processor, cause the system to:
present a first set of instructions to a subject regarding a first test;
receive an first input to initiate a first test based on the first set of instructions;
initiate a timer;
present a first stimulus to the subject auditorily via the speaker;
receive a first response from the subject auditorily via the microphone;
determine, based on the first set of instructions and a first answer key, if the first response is correct;
after determining that a threshold score has been achieved, present a second set of instructions to the subject regarding a second test, the second set of instructions being different than the first set of instructions;
receive a second input to initiate the second test based on the second set of instructions;
initiate the timer simultaneously with the received second input;
present a second stimulus to the subject auditorily via the speaker;
receive a second response from the subject auditorily via the microphone;
determine, based on the second set of instructions and a second answer key, if the second response is correct;
calculating results of at least one of the first test and the second test; and
displaying the results on a graphic user interface (GUI).

17. The system as recited in claim 16, further comprising instructions that, when executed by the at least one processor, cause the system to:
determine that the first response and/or the second response is correct; and
add a point to a score.

18. The system as recited in claim 16, further comprising instructions that, when executed by the at least one processor, cause the system to:
adjust a rate at which stimuli are presented based on a speed at which responses are received.

19. The system as recited in claim 16, further comprising instructions that, when executed by the at least one processor, cause the system to:
determine that the threshold score has not been achieved;
stopping the timer; and
prompting the subject to restart the first test.

20. A method of testing processing speed in humans, comprising:
presenting a subject with a set of instructions including a rule;
receiving an input from the subject to initiate a test;
initiating a timer simultaneously with the received input;
presenting an auditory stimulus, simultaneously with the received input, to the subject;
receiving an auditory response from the subject; and
determining, based on the set of instructions and an answer key, if the response is correct.
